(19) United States
(12) Patent Application Publication
(10) Pub. No.: US 2016/0340675 A1
(43) Pub. Date: Nov. 24, 2016

(54) MICRORNA MODULATION OF ARTHEROSCLEROSIS AND USES THEREOF

(71) Applicant: Vanderbilt University, Nashville, TN (US)

(72) Inventors: Kasey Vickers, Franklin, TN (US); Carrie Wiese, Nashville, TN (US); Valentina Kon, Nashville, TN (US); Zhi-Qi Xu, Nashville, TN (US)

(21) Appl. No.: 15/161,773

(22) Filed: May 23, 2016

Related U.S. Application Data
(60) Provisional application No. 62/164,837, filed on May 21, 2015.

(51) Int. Cl. C12N 15/113; C12Q 1/68

(52) U.S. Cl. CPC C12N 15/113; C12Q 1/6883; C12N 2310/113; C12N 2310/3231; C12Q 2600/178; C12Q 2600/158; C12Q 2600/118; C12N 2320/30; C12N 2310/3513

(57) ABSTRACT

The presently disclosed subject matter provides anti-miRNAs for the treatment of diseases associated with atherogenesis and methods of treatment, and diagnosis and/or prognosis of these diseases using lipoprotein-associated miRNAs.

### RELATED APPLICATION

This application claims priority from U.S. Provisional Patent Application No. 62/164,837, filed May 21, 2015, the entire disclosure of which is incorporated herein by this reference.

### GOVERNMENT INTEREST

This invention was made with government support under Grant Nos. K22HL113039, P01HL116263, and T32HL069765 awarded by the National Institutes of Health. The government has certain rights in the invention.

### TECHNICAL FIELD

The presently-disclosed subject matter relates to characterization and/or prediction of conditions, and evaluation of treatment and/or progression of conditions, involving a determination of the amount of one or more lipoprotein-associated miRNAs in a biological sample. The presently-disclosed subject matter also related to a method of treating such conditions, which include conditions associated with increased atherogenesis and cardiometabolic diseases. The presently-disclosed subject matter further relates to a unique method of treating artherosclerosis using a composition including inhibitors of miRNAs, such as anti-microRNAs (anti-miRs, anti-miRNAs, anti-miRNA oligonucleotide (AMOs)).

### INTRODUCTION

The identification of markers suitable for early detection, diagnosis, and treatment of atherosclerosis holds great promise to improve the clinical outcome of subjects. Atherosclerosis and cardiovascular disease are commonly known as the ‘silent killer.’ Thus, early detection, diagnosis and treatment is especially important for subjects presenting with vague or no symptoms. Hypercholesterolemia, homocysteine, oxidative stress, and hyperglycemia have been recognized as the major risk factors for atherogenesis. Additionally, there are many cardiometabolic diseases associated with increased atherogenesis and cardiovascular disease. For example, chronic kidney disease patients have accelerated atherosclerosis and increased cardiovascular events. There exists a need for novel ways to identify and treat patients with atherosclerosis, metabolic syndrome, rheumatoid arthritis, dyslipidemia, chronic kidney disease, diabetes, autoimmune disorders, and many other cardiometabolic diseases that are associated with increased atherogenesis and cardiovascular disease.

Accordingly, a need persists for the development of methods to predict and identify atherosclerosis and methods to treat subjects with atherosclerosis, dyslipidemia, chronic kidney disease, diabetes, autoimmune disorders, and many other cardiometabolic diseases that are associated with increased atherogenesis and cardiovascular disease. See, for example Andreas Schober, Maliheh Nazari-Jahantigh& Christian Weber, “MicroRNA-mediated mechanisms of the cellular stress response in atherosclerosis”, Nature Reviews Cardiology, 2015 and Clément Cochain & Alma Zernecke, “Noncoding RNAs in vascular inflammation and atherosclerosis: recent advances toward therapeutic applications” Current Opinion in Lipidology, 2014, the contents of which are incorporated in their entirety.

Treatment of existing atherosclerosis to induce regression, attenuate atherosclerosis, or prevent future atherosclerotic disease would significantly improve the outcome of subjects with cardiometabolic and other diseases where atherosclerosis is present.

### SUMMARY

The presently disclosed subject matter includes a method of inhibiting one or more miRNAs in a subject by administering to the subject an agent that inhibits miR-489, miR-92a or a combination thereof. In some embodiments, the agent is associated with a lipoprotein.

The method of inhibiting one or more miRNAs can be administered to a subject that has increased atherogenesis. In some embodiments, atherogenesis is altered when the miRNAs are inhibited. In some embodiments, the subject has atherosclerosis or has been identified as being at risk of developing atherosclerosis. In some embodiments, the subject has chronic kidney disease. In some embodiments, the subject has one or more conditions selected from atherosclerosis, dyslipidemia, diabetes, an autoimmune disorder, such as lupus, and a cardiometabolic disease associated with increased atherogenesis and cardiovascular disease. Conditions associated with increased atherogenesis are contemplated for treatment with the presently disclosed methods.

In some embodiments, the miRNA is associated with a lipoprotein. In some embodiments, the lipoprotein is selected from the group consisting of HDL, LDL, and VLDL and subfractions of lipoproteins. In some embodiments, the lipoprotein-associated miRNA is miR-489, miR-92a or a combination thereof.

In some embodiments, the agent inhibiting the miRNA is a lipoprotein-associated locked-nucleic acid inhibitor. In some embodiments, the agent utilized is one or more non-naturally occurring nucleic acid molecules that is an anti-miRNA oligonucleotide, which reduces miR-489 expression, miR-92a expression or a combination thereof. In some embodiments, the agent is a nucleic acid molecule comprising at least 10, 11, 12, 13, 14, 15, 16, 17, 18, 19, 20, or 21 consecutive nucleotides of the complement of a sequence selected from the group consisting of SEQ ID NO: 1 and SEQ ID NO:2. In some embodiments, the agent is a nucleic acid molecule consisting of a sequence selected from the group consisting of linked nucleic acid anti-miR-489 and linked nucleic acid anti-miR-92a.

The methods including an agent inhibiting the miRNAs of the presently disclosed subject matter can, in some embodiments, down-regulate, or decrease, the expression of the miRNA(s). In some embodiments, the miRNA expression is down-regulated in vascular endothelial cells and/or vascular tissues.

A method of determining an amount of one or more lipoprotein-associated miRNAs in a sample are also disclosed herein. In some embodiments, the method of determining an amount of lipoprotein-associated miRNAs include the steps of contacting the sample with a probe that specifically-binds each of the one or more lipoprotein-associated miRNAs, determining an amount of each of the probe-bound miRNAs, wherein the lipoprotein is selected from HDL, LDL, and VLDL and the one or more lipoprotein-associated miRNAs are selected from the group consisting of miR-489 and miR92a.

In some embodiments, a method of predicting whether a subject has or is at risk of developing a condition associated with increased atherogenesis is provided. The steps of predicting whether as subject has or is at risk of developing a condition includes steps of: providing a biological sample from the subject; contacting the sample with a probe that specifically-binds each of one or more miRNAs, determining an amount of each of the probe-bound miRNAs, and predicting that the subject has or is at risk of developing the condition when there is an increased level of the one more lipoprotein-associated miRNAs as compared to a control level and the one or more lipoprotein-associated miRNAs are selected from the group consisting of miR-489 and miR-92a. In some embodiments, the increased level of miRNAs are in circulation and/or vasculature and/or vascular cells. In some embodiments, the condition is atherosclerosis. In some embodiments, the subject has chronic kidney disease. In some embodiments, the condition is selected from dyslipidemia, diabetes, an autoimmune disorder, such as lupus, and a cardiometabolic disease associated with increased atherogenesis and cardiovascular disease. With regard to the biological sample utilized, the sample can be, in some instances, blood or plasma.

In some embodiments, the probe utilized includes a nucleic acid molecule having at least 10, 11, 12, 13, 14, 15, 16, 17, 18, 19, 20, or 21 consecutive nucleotides of selected from the group consisting of SEQ ID NO; 1 and SEQ ID NO: 2 or a complement thereof. In some embodiments, the probe comprises a nucleic acid molecule consisting of a sequence selected from the group consisting of antisense miR-489 and antisense miR-92a.

In some embodiments, the probe further comprises a label. The label is, in some embodiments, a radioisotope, a fluorescent label, a chemiluminescent label, or an enzymatic label.

In some embodiments, the method includes a step of administering to the subject an agent that inhibits the one or more lipoprotein-associated miRNA, when the subject is predicted as having or being at risk of developing the condition, the agent being an inhibitor of selected from the group consisting of miR-489 and miR-92a, the agent associated with a lipoprotein.

In some embodiments diagnosing a condition in a subject occurs prior to administering an agent, where diagnosing includes obtaining a blood or plasma sample from the subject; and isolating lipoprotein from the blood or plasma. In some embodiments, the diagnosing also includes detecting whether miR-489 and/or miR-92a are present in the isolated lipoproteins and may optionally include diagnosing the subject with the condition when miR-489 and/or miR-92a are present in the isolated lipoproteins at increased levels relative to a control. In some embodiments, the method includes diagnosing the subject with the condition when miR-489 and/or miR-92a are present in the isolated lipoproteins at increased levels relative to a control.

In some embodiments, a method of detecting lipoprotein-associatedmiRNAs in a sample, comprising: contacting the sample with a probe that specifically-binds each of the one or more lipoprotein-associated miRNAs, and determining an amount of each of the probe-bound miRNAs. In some embodiments, the lipoprotein is selected from HDL, LDL, and VLDL and subfractions of lipoprotein, and the one or more lipoprotein-associated miRNAs are selected from the group consisting of miR-489 and miR-92a In some embodiments, the sample is a lipoprotein sample isolated from a blood or plasma sample from a subject.

A method for evaluating treatment efficacy and/or progression of a condition associated with increased atherogenesis in a subject is provided herein. In some embodiments, the method includes the steps of contacting a biological sample of the subject with a probe that specifically-binds each of one or more miRNAs; and comparing the amount of probe-bound miRNAs to a reference, wherein the one or more miRNAs are selected from the group consisting of miR-489 and miR-92a. In some embodiments, the reference comprises a sample collected prior to initiation of treatment for the condition and/or onset of the condition and the biological sample comprises a biological sample collected after initiation of the treatment or onset.

In some embodiments of the methods disclosed herein, a reagent is used. Also disclosed herein, in some embodiments, are a kit comprising a reagent and one or more probes that specifically-binds each of one or more miRNAs, optionally associated with a lipoprotein, the one or more miRNAs are selected from the group consisting of miR-92a and miR-489.

In some embodiments, a composition is provided. In some embodiments, the composition includes one or more agents that each inhibit a lipoprotein-associated miRNA, wherein the lipoprotein is selected from the group consisting of HDL, LDL, and VLDL and the one or more agents are selected from the group consisting of: a non-naturally occurring anti-miRNA 92a, and a non-naturally occurring anti-miRNA-489. The one or more agents, in some embodiments, are locked-nucleic acid inhibitors. In some embodiments, the one or more agents include a non-naturally-occurring nucleic acid molecule comprising at least 10, 11, 12, 13, 14, 15, 16, 17, 18, 19, 20, or 21 consecutive nucleotides of the complement of SEQ ID NO: 1, or a non-naturally-occurring nucleic acid molecule comprising at least 10, 11, 12, 13, 14, 15, 16, 17, 18, 19, 20, or 21 consecutive nucleotides of the complement of SEQ ID NO: 2.

### BRIEF DESCRIPTION OF THE DRAWINGS

An understanding of the features and advantages of the embodiments of the presently-disclosed subject matter will be obtained by reference to the following detailed description that sets forth illustrative embodiments, in which the principles of the invention are used, and the accompanying drawings of which:

FIG. 1 includes a diagram demonstrating HDL mediated delivery of miRNA inhibitor to the endothelium with the end goal of inhibiting atherosclerosis development. The miRNA inhibitor is a lock nucleic acid (LNA) designed to specifically inhibit the miRNA. B.) Diagram of chronic kidney disease mouse model, which includes ⅚ nephrectomy (⅚Nx) surgery to remove ⅚ of the kidney mass. This project specifically involves ⅚ nephrectomy on a well-studied atherosclerosis mouse model, Apoe^{-/-} mice.

FIG. 2 compares the change in miR-92a and miR-489 in the mouse model with reduced HDL levels (Apoe−/− mice) with and without ⅚ Nx to wild type (WT) mice. miR-489 and miR-92a levels were increased in both wild type and Apoe−/− aortic endothelium after ⅚ Nx compared to controls without ⅚ Nx.

FIG. 3 includes quantification of miR-92a and miR-489 in the aorta endothelium for Apoe^{-/-} mice with sham or ⅚nx surgeries. The data is the same as in FIG. 2, but regraphed to demonstrate the difference specifically in the Apoe−/− mouse background. N=14-21. Results were based on Real-time PCR showing elevation of the miRNAs in chronic kidney disease-associated atherosclerosis. Significance was determined by Mann-Whitney nonparametric test.

FIG. 4 includes HDL-LNA treatment procedure and the results of miR489 (KCV) and miR92(a) levels quantified in aortic endothelium RNA by real-time PCR. miR489 and miR-92a levels were decreased in WT mice with ⅚ nephrectomy (⅚Nx) after injections in the retro-orbital plexus with HDL complexed with locked-nucleic acid inhibitors against miR-92a or miR-489 (4 mg HDL+20 m/kg LNA) compared to saline (control) or HDL (4 mg) injections. This study was a proof-of-concept to confirm knockdown of miRNA in the endothelium after treatment with HDL+LNA. *P<0.05.

FIG. 5 includes an experimental study diagram outlining the time course of one of the exemplary experiments. Apoe^{-/-} mice undergo surgery, sham or ⅚nx, and over a 7 week period the mice develop more severe atherosclerotic lesions due to the decrease in kidney function. At 7 weeks post, a subset of mice are taken as baseline control to measure the atherosclerotic lesion at the start of the treatment. At this 7 week time point, the other mice are treated with a single intravenous injection of 4 mg HDL, 4mg HDL+20 mg/kg LNA-92a, 4 mg HDL+20 mg/kg LNA-489, or 4 mg HDL+20 mg/kg LNA-92a+20 mg/kg LNA-489. The mice are sacrificed for tissue collection 1 week after this single treatment.

FIG. 6 provides results of real-time PCR quantification of miR-92a and miR-489 in the aorta endothelium of HDL+/−LNA treated Apoe^{-/-} mice demonstrate that the LNAs reduce the miRNAs in the endothelium. N=6-13. Statistical significance determined by one-way ANOVA.

FIG. 7 includes images of cross sections of the aortic sinus stained with oil red o to visualize the neutral lipids within the atherosclerotic lesions.

FIG. 8 includes results of the calculated total lesion area for mice treated with ⅚ Nx, HDL, LNA-489 and LNA-92a. The lesion area is the cumulative oil red o stained area quantified across 15 cross-sections per mouse. N=5-11. Statistical significance was determined by one-way ANOVA.

FIG. 9 includes data from the aortas from a small cohort stained with Sudan IV en face to quantify the lesions throughout the length of the aortas. The lesion area as a percent of the whole aorta was quantified with representative images for each experimental group. N=3-4. Statistical significance was determined by one-way ANOVA.

FIG. 10 includes A) The necrotic area percent of the lesion was quantified from hematoxylin and eosin (H&E) stained cross-sections. B) The lesion collagen content was visualized and quantified using aniline blue staining. C) The aniline blue stained images are representative images for each experimental group. N=4-9 Statistical significance was determined by one way ANOVA.

FIG. 11 includes charts evaluating blood physiology including total plasma cholesterol and blood urea nitrogen after treatment with LNA-489, LNA-92a, HDL, or ⅚ Nx. The total plasma cholesterol was quantified by a colormetric assay using sodium citrate collected plasma. The blood urea was quantified by a colorimetric assay using serum.

FIG. 12 includes Volcano plots of significantly differential abundant mRNAs in the aorta endothelium of the HDL and HDL+LNA treated mice compared to ⅚ nx saline control. Red is 1.5 fold or greater decrease. Green is 1.5 fold or greater increase. The differentially regulated mRNAs were identified by total RNA Illumina sequencing. N=4.

FIG. 13 includes a graph showing an 8.5 fold increase in HDL-miR-92a levels from chronic kidney disease subjects compared to normal controls.

### DESCRIPTION OF THE SEQUENCE LISTING

SEQ ID NO 1 is an exemplary RNA sequence for miR-489 from homo sapiens.

SEQ ID NO 2 is an exemplary RNA sequence for miR-92a from homo sapiens.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

Although any methods, devices, and materials similar or equivalent to those described herein can be used in the practice or testing of the presently-disclosed subject matter, representative methods, devices, and materials are now described.

The presently-disclosed subject matter includes micro-RNA-489 and micro-RNA-92a modulation of cardiometabolic diseases and uses thereof. This invention aims to address unmet needs in the field of atherosclerosis subsequent to chronic kidney disease and other diseases where atherosclerosis is independent of chronic kidney disease, such as in lupus. Delivery of anti-miR-489 or anti-mi-R92a or a combination of both, associated with a lipoprotein, causes regression of the plaque buildup seen in the disease. This treatment will be particularly useful to attenuate atherosclerosis in subject who cannot tolerate or take statin class drugs. This invention can be used to treat and prevent atherosclerosis.

While miRNAs have been previously identified in connection with certain diseases, the present inventors have demonstrated that, lipoprotein-associated miRNAs, including HDL-LDL- and VLDL-associated miRNAs, can be useful for predicting a condition associated with increased atherogenesis.

In particular, as disclosed herein, lipoprotein-associated mi-RNA-489 and mi-RNA-92a are useful for predicting a condition associated with increased atherogenesis, such as chronic kidney disease, atherosclerosis, including atherosclerosis in connection with chronic kidney disease, dyslipidemia, diabetes, an autoimmune disorder, such as lupus, and other cardiometabolic diseases associated with increased atherogenesis and cardiovascular disease.

As also disclosed herein, anti-miRNA 92a, or anti-miR-489, or a combination of the two can be used in a composition for the treatment of and/or in a method of treating a condition associated with increased atherogenesis, such as atherosclerosis, chronic kidney disease, dyslipidemia, diabetes, an autoimmune disorder, such as lupus, and other cardiometabolic diseases associated with increased atherogenesis and cardiovascular disease. In some embodiments of the presently-disclosed subject matter, treatment of atherosclerosis can be achieved by delivering inhibitors of mi-RNA 92a, mi-RNA 489, or a combination of mi-RNA 92a and mi-RNA 489. Further, the present inventors have discovered that anti-miR-489 and/or anti-miR-92a regulated gene expression in vascular tissues and increases anti-atherogenic gene expression in vascular endothelial cells.

As used herein, the terms “treatment” or “treating” are inclusive of prophylactic treatment and therapeutic treatment, and include curing or substantially curing a condition, ameliorating at least one symptom of the condition, and preventing the progression of at least one symptom of the condition. As would be recognized by one or ordinary skill in the art, treatment that is administered prior to clinical manifestation of a condition is prophylactic (i.e., it protects the subject against developing the condition). If the treatment is administered after manifestation of the condition, the treatment is therapeutic (i.e., it is intended to diminish, ameliorate, control, or maintain the existing condition and/or side effects associated with the condition).

The terms relate to medical management of a subject with the intent to substantially cure, ameliorate, stabilize, or substantially prevent a condition of interest (e.g., disease, pathological condition, or disorder), including but not limited to prophylactic treatment to preclude, avert, obviate, forestall, stop, or hinder something from happening, or reduce the severity of something happening, especially by advance action.

As such, the terms treatment or treating include, but are not limited to: inhibiting the progression of a condition of interest; arresting or preventing the development of a condition of interest; reducing the severity of a condition of interest; ameliorating or relieving symptoms associated with a condition of interest; causing a regression of the condition of interest or one or more of the symptoms associated with the condition of interest; and preventing a condition of interest or the development of a condition of interest.

As disclosed herein, the present inventors have discovered that HDL transfers functional miRNAs between cells and tissues within intercellular communication networks. The lipoprotein-miRNA communication is altered in chronic kidney disease and contributes to atherosclerosis. It was surprisingly discovered that the lipoprotein-miRNA communication can be manipulated to prevent atherosclerosis progression and induce lesion regression. Anti-miR-92a, or anti-miR-489, or a combination of the two can be utilized as therapeutic or preventative agents in accordance with the teachings of the present invention. Further, a method of treating atherosclerosis can be achieved by delivering inhibitors of mi-R92a, mi-R489, or a combination of mi-R92a and mi-R489. A method of measuring the lipoprotein-associated mi-R92a, mi-R 489 as a biomarker for chronic kidney disease progression, response to treatment, or atherosclerosis is also provided herein.

In some embodiments of the presently disclosed subject matter, a method for characterizing a disease in a subject is provided. Characterizing can include providing a diagnosis, prognosis, and/or theranosis of the disease. In some embodiments, the method can include isolating lipoprotein associated miRNAs from a biological sample of the subject, determining an amount of one or more miRNAs in sample, and comparing the amount of the one or more miRNAs to a reference, wherein the disease is characterized based on a measurable difference in the amount of the one or more miRNAs as compared to a control. For example, in some embodiments the subject can be diagnosed as having the disease or risk thereof if there is a measurable difference in the amount of the one or more miRNAs from the sample as compared to a reference.

In some embodiments of the presently-disclosed subject matter, a method for evaluation of treatment efficacy and/or progression of a disease in a subject is provided. In some embodiments, the method can involve obtaining a biological sample of the subject, determining an amount of one or more miRNAs in the sample, and determining any measurable change in the amounts of the one or more miRNAs to thereby evaluate treatment efficacy and/or progression of atherosclerosis in the subject. In some embodiments, the biological sample can include a first biological sample collected prior to initiation of treatment for the disease and/or onset of the disease and a second biological sample collected after initiation of the treatment or onset. In some embodiments, the method can also include selecting a treatment or modifying a treatment for the disease based on the amount of the one or more miRNAs determined.

In some embodiments of the presently disclosed subject matter, a method of treating atherosclerosis or cardiovascular disease in a subject is provided. In some embodiments, the method can involve obtaining a biological sample of the subject, determining an amount of one or more miRNAs in the sample, and determining any measurable change in the amounts of the one or more miRNAs to thereby evaluate treatment efficacy and/or progression of atherosclerosis in the subject. In some embodiments, the biological sample can include a first biological sample collected prior to initiation of treatment for the disease and/or onset of the disease and a second biological sample collected after initiation of the treatment or onset. In some embodiments, the method can also include selecting a treatment or modifying a treatment for the disease based on the amount of the one or more miRNAs determined.

The disease of interest can be, for example, a cardiometabolic disease. In some embodiments, the cardiometabolic disease is chronic kidney disease. In another example, the disease is lupus. In another example, the disease can be any disease associated with increased atherogenesis and cardiovascular disease. Some of the diseases associated with increased atherogenesis include dyslipidemia, chronic kidney disease, metabolic syndrome, rheumatoid arthritis, diabetes, and autoimmune disorders. However, one skilled in the art will recognize other diseases associated with atherogenesis and atherosclerosis. The presently disclosed subject matter is applicable to diseases associated with atherogenesis and atherosclerosis.

In some embodiments of the presently disclosed subject matter, a method of increasing anti-atherogenic gene expression is disclosed. In some embodiments, the increase in anti-atherogenic gene expression occurs in vascular endothelial cells. In some embodiments the method comprises administering intravenously an antisense molecule against one or more miRNAs to reduce miRNA expression. In some embodiments, the presently described invention provides a method of increasing anti-atherogenic gene expression, the method comprising contacting a subject with an agent against one or more miRNAs from the group consisting of: miR-92a and miR-489 in a dose effective to increase anti-atherogenic gene expression. In some embodiments, the presently described invention provides a method for treating a disease in a subject, the method comprising contacting a subject with an agent against one or more miRNAs from the group consisting of: miR-92a and miR-489. In some preferred embodiments, the mi-RNA agent is a locked-nucleic acid.

In some embodiments of the presently disclosed subject matter, a method of regulating anti-atherogenic gene expression is disclosed. In some embodiments, the regulating of anti-atherogenic gene expression occurs in vascular endothelial cells. In some embodiments the method comprises administering intravenously an antisense molecule against one or more miRNAs. In some embodiments, the presently described invention provides a method of regulating anti-atherogenic gene expression, the method comprising contacting a subject with an agent against one or more miRNAs from the group consisting of: miR-92a and miR-489 in a dose effective to regulate anti-atherogenic gene expression. In some embodiments, the presently described invention provides a method for treating a disease in a subject, the method comprising contacting a subject with an agent against one or more miRNAs from the group consisting of: miR-92a and miR-489. In some preferred embodiments, the mi-RNA agent is a locked-nucleic acid.

In some embodiments, the one or more RNAs include one or more miRNAs selected from the group consisting of: miR-92a and miR-489. In some embodiments a change in expression of the one or more miRNAs as compared to the reference is indicative of a change in the level of atherosclerosis.

In some embodiments, the miRNAs, anti-miRNAs, and interference to miRNAs are optionally associated with a lipoprotein. In some embodiments, the lipoprotein is HDL. The term “lipoprotein” includes high-density lipoprotein (HDL), low-density lipoprotein (LDL), and very low-density lipoprotein (VLDL), and subfractions of lipoproteins. The miRNAs and miRNA inhibitors can optionally be associated, or complexed, with lipoproteins, in some embodiments, by providing the miRNA and lipoprotein together when administered.

In some embodiments, the methods of the invention comprise providing a biological sample from a subject and measuring miRNAs from the biological sample. The biological sample can be a bodily fluid such as described herein, e.g., plasma or serum. An amount of one or more of the RNAs is then determined and compared to one or more miRNA control levels. The subject can then be diagnosed with having or being at risk of disease if there is a measurable difference in the amount of the one or more miRNAs as compared to the one or more miRNA control levels. The levels of the one or more miRNAs can also be used to provide a prognosis or a theranosis, such as to classify the subject as a likely responder or non-responder to a treatment or to monitor the efficacy of a treatment over time. As such, in some embodiments, methods can include predicting response to a treatment in a subject, or predicting non-response of a treatment in a subject. The control levels can be the levels of the one or more miRNAs in a control sample that does not have or is not at risk of having disease, e.g., the control sample can be from a healthy subject. When monitoring one or more miRNA levels over time, a control can also be the level of the one or more miRNAs at a different time point. For example, a decrease in the level of one or more miRNA in a subject over time may indicate a response to a treatment.

“Making a diagnosis” or “diagnosing,” as used herein, are further inclusive of making a prognosis, which can provide for predicting a clinical outcome (with or without medical treatment), selecting an appropriate treatment (or whether treatment would be effective), or monitoring a current treatment and potentially changing the treatment, based on the measure of mi-RNA levels. Diagnostic testing that involves treatment, such as treatment monitoring or decision making can be referred to as “theranosis.” Further, in some embodiments of the presently disclosed subject matter, multiple determination of amounts of one or more miRNAs over time can be made to facilitate diagnosis (including prognosis), evaluating treatment efficacy, and/or progression of a disease. A temporal change in one or more miRNA levels (i.e., miRNA amounts in a biological sample) can be used to predict a clinical outcome, monitor the progression of the disease, and/or efficacy of administered disease therapies. In such an embodiment for example, one could observe a decrease in the amount of particular miRNAs in a biological sample over time during the course of a therapy, thereby indicating effectiveness of treatment.

In some embodiments, the presently disclosed subject matter provides a method for treating a disease in a subject. In some embodiments, the treatment is administered parenterally. It is contemplated treatment could also be by inhalation, digestion, and other enteral routes. In some embodiments the method comprises administering intravenously an antisense molecule against one or more miRNAs to reduce miRNA expression. In some embodiments, the presently described invention provides a method of altering atherogenesis; the method includes contacting a subject with an agent against one or more miRNAs from the group consisting of: miR-92a and miR-489 in a dose effective to alter the atherogenesis. In some embodiments, the presently described invention provides a method for treating a disease in a subject, the method comprising contacting a subject with an agent against one or more miRNAs from the group consisting of: miR-92a and miR-489. In some preferred embodiments, the mi-RNA agent is a locked-nucleic acid. In some embodiments, the atherogenesis of the cell is down-regulated as a result of the treatment.

The term “effective amount” means that quantity of a miRNA inhibitor which has a positive therapeutic effect for treating or preventing the condition which affects the subject. Such amount is that which inhibits the miRNA; in other words, a miRNA inhibiting amount. In some embodiments, mice were treated with 4 mg of HDL and 20 mg of miRNA inhibitor per kilogram of body weight. While the dosage in other mammals will vary, those skill in the art will appreciate such variances are calculable and optimized through routine experimentation.

The presently disclosed subject matter further provides in some embodiments a method for theranostic testing, such as evaluating treatment efficacy and/or progression of a disease in a subject. In some embodiments, the method comprises providing a series of biological samples over a time period from the subject; determining an amount of one or more of the miRNAs in each of the biological samples from the series; and determining any measurable change in the amounts of the one or more miRNAs in each of the biological samples from the series to thereby evaluate treatment efficacy and/or progression of the disease in the subject. Any changes in the amounts of measured miRNAs over the time period can be used to predict clinical outcome, determine whether to initiate or continue the therapy for the disease, and whether a current therapy is effectively treating the disease. For example, a first time point can be selected prior to initiation of a treatment and a second time point can be selected at some time after initiation of the treatment. miRNA levels can be measured in each of the samples taken from different time points and qualitative and/or quantitative differences noted. A change in the amounts of one or more of the measured miRNA levels from the first and second samples can be correlated with prognosis, theranosis, determining treatment efficacy, and/or progression of the disease in the subject.

The terms “correlated” and “correlating,” as used herein in reference to the use of diagnostic and prognostic miRNA levels associated with a disease, refer to comparing the presence or quantity of the miRNA levels in a subject to its presence or quantity in subjects known to suffer from a disease, or in subjects known to be free of the disease, i.e. “normal subjects” or “control subjects.” For example, a level of one or more miRNAs in a biological sample can be compared to a miRNA level for each of the specific miRNAs tested and determined to be correlated with a disease. The sample's one or more miRNA levels is said to have been correlated with a diagnosis; that is, the skilled artisan can use the miRNA level(s) to determine whether the subject suffers from the disease and respond accordingly. Alternatively, the sample's miRNA level(s) can be compared to control miRNA level(s) known to be associated with a good outcome (e.g., the absence of disease), such as an average level found in a population of normal subjects.

In certain embodiments, a diagnostic or prognostic miRNA level is correlated to a disease by merely its presence or absence. In other embodiments, a threshold level of a diagnostic or prognostic miRNA level can be established, and the level of the miRNA in a subject sample can simply be compared to the threshold level.

As noted, in some embodiments, multiple determinations of one or more diagnostic or prognostic miRNA levels can be made, and a temporal change in the levels can be used to determine a diagnosis or prognosis. For example, specific miRNA level(s) can be determined at an initial time, and again at a second time. In such embodiments, an increase in the miRNA level(s) from the initial time to the second time can be diagnostic of the disease, or a given prognosis. Likewise, a decrease in the miRNA level(s) from the initial time to the second time can be indicative of the disease, or a given prognosis. Furthermore, the degree of change of one or more miRNA level(s) can be related to the severity of the disease and/or timeline of disease progression and future adverse events.

The skilled artisan will understand that, while in certain embodiments comparative measurements can be made of the same miRNA level(s) at multiple time points, one can also measure given miRNA level(s) at one time point, and second miRNA level(s) at a second time point, and a comparison of these levels can provide diagnostic information.

A subject at risk or at elevated risk of developing a condition includes subjects identified as having certain risk factors or a combination of risk factors. Risk factors may include, for example, elevated inflammation markers such as C Reactive protein, hypertriglyceridemia, genetic factors and/or family history, obesity, high blood pressure, unhealthy blood cholesterol levels (high LDL-cholesterol levels or particle numbers and/or low HDL-cholesterol levels or particle numbers), age, metabolic syndrome, or any other factor associated with developing a condition associated with increased atherogenesis or atherosclerosis. A subject at risk may also have a condition or be at elevated risk for a condition that is associated with increased atherogenesis or atherosclerosis, such as, for example, chronic kidney disease, autoimmune disorders, diabetes or cardiometabolic disease.

The phrase “determining the prognosis” as used herein refers to methods by which the skilled artisan can predict the course or outcome of a condition in a subject. The term “prognosis” can refer to the ability to predict the course or outcome of a condition with up to 100% accuracy, or predict that a given course or outcome is more or less likely to occur based on the presence, absence or levels of a biomarker. The term “prognosis” can also refer to an increased probability that a certain course or outcome will occur; that is, that a course or outcome is more likely to occur in a subject exhibiting a given condition, when compared to those individuals not exhibiting the condition. For example, in individuals not exhibiting the condition (e.g., not expressing the miRNA level(s) or expressing miRNA level(s) at a reduced level), the chance of a given outcome (e.g., suffering from disease) may be very low (e.g., <1%), or even absent. In contrast, in individuals exhibiting the condition (e.g., expressing the miRNA level(s) or expressing miRNA level(s) at a level greatly increased over a control level), the chance of a given outcome (e.g., suffering from a form/stage of disease) may be higher. In certain embodiments, a prognosis is about a 5% chance of a given expected outcome, about a 7% chance, about a 10% chance, about a 12% chance, about a 15% chance, about a 20% chance, about a 25% chance, about a 30% chance, about a 40% chance, about a 50% chance, about a 60% chance, about a 75% chance, about a 90% chance, or about a 95% chance.

The skilled artisan will understand that associating a prognostic indicator with a predisposition to an adverse outcome can be performed using statistical analysis. For example, miRNA level(s) (e.g., quantity of one or more miRNAs in a sample) of greater or less than a control level in some embodiments can signal that a subject is more likely to suffer from a disease than subjects with a level less than or equal to the control level, as determined by a level of statistical significance. Additionally, a change in miRNA level(s) from baseline levels can be reflective of subject prognosis, and the degree of change in marker level can be related to the severity of adverse events. Statistical significance is often determined by comparing two or more populations, and determining a confidence interval and/or a p value. See, e.g., Dowdy and Wearden, Statistics for Research, John Wiley & Sons, New York, 1983, incorporated herein by reference in its entirety. Exemplary confidence intervals of the present subject matter are 90%, 95%, 97.5%, 98%, 99%, 99.5%, 99.9% and 99.99%, while exemplary p values are 0.1, 0.05, 0.025, 0.02, 0.01, 0.005, 0.001, and 0.0001. When performing multiple statistical tests, e.g., determining differential expression of a panel of miRNA levels, p values can be corrected for multiple comparisons using techniques known in the art.

In other embodiments, a threshold degree of change in the level of a prognostic or diagnostic miRNA level(s) can be established, and the degree of change in the level of the indicator in a biological sample can simply be compared to the threshold degree of change in the level. A preferred threshold change in the level for miRNA level(s) of the presently disclosed subject matter is about 5%, about 10%, about 15%, about 20%, about 25%, about 30%, about 50%, about 60%, about 75%, about 100%, or about 150%. In yet other embodiments, a “nomogram” can be established, by which a level of a prognostic or diagnostic indicator can be directly related to an associated disposition towards a given outcome. The skilled artisan is acquainted with the use of such nomograms to relate two numeric values with the understanding that the uncertainty in this measurement is the same as the uncertainty in the marker concentration because individual sample measurements are referenced, not population averages.

The identity and relative quantity of miRNAs in a sample can be used to provide miRNA profiles for a particular sample. An miRNA profile for a sample can include information about the identities of miRNAs contained in the sample, quantitative levels of miRNAs contained in the sample, and/or changes in quantitative levels of miRNAs relative to another sample. For example, an miRNA profile for a sample can include information about the identities, quantitative levels, and/or changes in quantitative levels of miRNAs associated with a particular disease.

Further with respect to the diagnostic methods of the presently disclosed subject matter, a preferred subject is a vertebrate subject. A preferred vertebrate is warm-blooded; a preferred warm-blooded vertebrate is a mammal. A mammal is most preferably a human. As used herein, the term “subject” includes both human and animal subjects. Thus, veterinary therapeutic uses are provided in accordance with the presently disclosed subject matter.

As such, the presently disclosed subject matter provides for the diagnosis of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses (including race horses), poultry, and the like.

As noted hereinabove, the presently disclosed subject matter provides for the determination of the amount of mi RNAs correlated with disease within biological fluids of a subject, and in particular, from serological samples from a subject, such as for example blood. This provides the advantage of biological samples for testing that are easily acquired from the subject. The amount of one or more miRNAs of interest in the biologic sample can then be determined using any of a number of methodologies generally known in the art and compared to miRNA control levels.

The “amount” of one or more miRNAs determined refers to a qualitative (e.g., present or not in the measured sample) and/or quantitative (e.g., how much is present) measurement of the one or more miRNAs. The “control level” is an amount (including the qualitative presence or absence) or range of amounts of one or more miRNAs found in a comparable biological sample in subjects not suffering from disease. As one non-limiting example of calculating the control level, the amount of one or more miRNAs of interest present in a normal biological sample (e.g., blood) can be calculated and extrapolated for whole subjects.

An exemplary methodology for measuring miRNA levels in a biological sample is microarray technique, which is a powerful tool applied in gene expression studies. The technique provides many polynucleotides with known sequence information as probes to find and hybridize with the complementary strands in a sample to thereby capture the complementary strands by selective binding.

The term “selective bind” or “specifically bind” as used herein refers to a measure of the capacity of a probe to hybridize to a target polynucleotide with specificity. Thus, the probe comprises a polynucleotide sequence that is complementary, or essentially complementary, to at least a portion of the target polynucleotide sequence. Nucleic acid sequences which are “complementary” are those which are base-pairing according to the standard Watson-Crick complementarity rules. As used herein, the term “complementary sequences” means nucleic acid sequences which are substantially complementary, as can be assessed by the same nucleotide comparison set forth above, or as defined as being capable of hybridizing to the nucleic acid segment in question under relatively stringent conditions such as those described herein. A particular example of a contemplated complementary nucleic acid segment is an antisense oligonucleotide. With regard to probes disclosed herein having binding affinity to miRNAs, the probe can be 100% complementary with the target polynucleotide sequence. However, the probe need not necessarily be completely complementary to the target polynucleotide along the entire length of the target polynucleotide so long as the probe can bind the target polynucleotide with specificity and capture it from the sample.

Nucleic acid hybridization will be affected by such conditions as salt concentration, temperature, or organic solvents, in addition to the base composition, length of the complementary strands, and the number of nucleotide base mismatches between the hybridizing nucleic acids, as will be readily appreciated by the skilled artisan. Stringent temperature conditions will generally include temperatures in excess of 30° C., typically in excess of 37° C., and preferably in excess of 45° C. Stringent salt conditions will ordinarily be less than 1,000 mM, typically less than 500 mM, and preferably less than 200 mM. However, the combination of parameters is much more important than the measure of any single parameter. Determining appropriate hybridization conditions to identify and/or isolate sequences containing high levels of homology is well known in the art. For the purposes of specifying conditions of high stringency, preferred conditions are a salt concentration of about 200 mM and a temperature of about 45° C.

Data mining work is completed by bioinformatics, including scanning chips, small RNA sequencing, real-time PCR-based arrays, signal acquisition, image processing, normalization, statistic treatment and data comparison as well as pathway analysis. As such, small RNA sequencing, microarray, and/or real-time PCR can profile hundreds and thousands of polynucleotides simultaneously with high throughput performance. Profiling analysis of miRNA and/or mRNA expression has successfully provided valuable data for gene expression studies in basic research. And the technique has been further put into practice in the pharmaceutical industry and in clinical diagnosis. With increasing amounts of miRNA data becoming available, and with accumulating evidence of the importance of miRNA in gene regulation, profiling becomes a useful technique for high through-put miRNA studies. High-throughput approaches for microRNA expression analysis, Methods Mol Biol. 1107:91-103 (2014), incorporated herein by reference, discusses such approaches.

The analysis of miRNA correlated with disease can be carried out separately or simultaneously with multiple polynucleotide probes within one test sample. For example, several probes can be combined into one test for efficient processing of a multiple of samples and for potentially providing greater diagnostic and/or prognostic accuracy. In addition, one skilled in the art would recognize the value of testing multiple samples (for example, at successive time points) from the same subject. Such testing of serial samples can allow the identification of changes in miRNA levels over time. Increases or decreases in miRNA levels, as well as the absence of change in levels, can provide useful information about the disease status.

In some embodiments, a panel consisting of polynucleotide probes that selectively miRNAs correlated with one or more diseases can be constructed to provide relevant information related to the diagnosis or prognosis of disease and management of subjects with disease. Such a panel can be constructed, for example, using 1, 2, 3, 4, 5, 6, 7, 8, 9, 10, 15, 20, 30, 40, 50, 75, 100, 150, 200, 250, 300, 400, 500, or 1,000 individual polynucleotide probes. In some cases, a panel comprises more than 1,000 individual polynucleotide probes. The analysis of a single probe or subsets of probes comprising a larger panel of probes could be carried out by one skilled in the art to optimize clinical sensitivity or specificity in various clinical settings. These include, but are not limited to ambulatory, urgent care, critical care, intensive care, monitoring unit, in-subject, out-subject, physician office, medical clinic, and health screening settings. Furthermore, one skilled in the art can use a single probe or a subset of additional probes comprising a larger panel of probes in combination with an adjustment of the diagnostic threshold in each of the aforementioned settings to optimize clinical sensitivity and specificity. The clinical sensitivity of an assay is defined as the percentage of those with the disease that the assay correctly predicts, and the specificity of an assay is defined as the percentage of those without the disease that the assay correctly predicts.

Several measurement platforms can determine relative miRNA abundance in biological samples using different technologies such as small RNA sequencing, amplificantion based profiling such as qRT-PCR and (microarray) hybridization. qRT-PCR can serve as a platform for single reverse PCR amplification experiments and for a large number of miRNAs in parallel, both by multiplexing and plate based arrays. Additionally, miRNA profiling strategies based on deep sequencing allow both the identification of novel miRNAs and relative quantification of miRNAs. A guide to “Performing Relative Quanittation of Gene Expression Using Real-Time Quantitative PCR” published by Applied Biosystems, incorporated herein by reference, provides an overview of RT-PCR and relative quantitation of gene expression.

Hybridization based miRNA profiling techniques by microarray are utilized in commercial kits such as Agilent miRNA microarray, Affymetrix custom microarray and Nanostring nCounter. Quantitative RT-PCR is utilized by commercial kits including Exiqon miRCury, TaqMan Array Human MicroRNA cards, TaqMan OpenArray Human MicroRNA panel, Qiagen miScript, Quanta Bioscences qScript, and WaferGen Smart Chip. Sequencing approaches, such as next-generation sequencing, are utilized by the Ilumina TruSeq and Life Technologies Ion Torrent.

In some embodiments, determining the amount of the one or more miRNAs comprises labeling the one or more miRNAs. The labeled miRNAs can then be captured with one or more polynucleotide probes that each selectively bind the one or more miRNAs.

As used herein, the terms “label” and “labeled” refer to the attachment of a moiety, capable of detection by spectroscopic, radiologic, or other methods, to a probe molecule. Thus, the terms “label” or “labeled” refer to incorporation or attachment, optionally covalently or non-covalently, of a detectable marker into/onto a molecule, such as a polynucleotide. Various methods of labeling polypeptides are known in the art and can be used. Examples of labels for polynucleotides include, but are not limited to, the following: radioisotopes, fluorescent labels, heavy atoms, enzymatic labels or reporter genes, chemiluminescent groups, biotinyl groups, predetermined polypeptide epitopes recognized by a secondary reporter (e.g., leucine zipper pair sequences, binding sites for antibodies, metal binding domains, epitope tags, etc.). In some embodiments, labels are attached by spacer arms of various lengths to reduce potential steric hindrance.

As used herein, the term “inhibitor” refers to a molecule that reduces the expression of miRNA. The reduction of the expression takes place through providing a molecule such as an antisense molecule, synthetic miRNA inhibitors, and transcription factor inhibitors. Synthetic miRNA inhibitors can include locked-nucleic acid sequences. An antisense molecule is a single stranded molecule having a sequence that is wholly or mostly reverse complementary to an miRNA and inhibits the function of an miRNA through hybridization.

The analysis of miRNA levels using polynucleotide probes can be carried out in a variety of physical formats as well. For example, the use of microtiter plates or automation can be used to facilitate the processing of large numbers of test samples. Alternatively, single sample formats could be developed to facilitate immediate treatment and diagnosis in a timely fashion.

In some embodiments, the plurality of polynucleotide probes are each bound to a substrate. In some embodiments, the substrate comprises a plurality of addresses. Each address can be associated with at least one of the polynucleotide probes of the array. An array is “addressable” when it has multiple regions of different moieties (e.g., different polynucleotide sequences) such that a region (i.e., a “feature” or “spot” of the array) at a particular predetermined location (i.e., an “address”) on the array will detect a particular target or class of targets (although a feature may incidentally detect non-targets of that feature). Array features are typically, but need not be, separated by intervening spaces. In the case of an array, the “target” miRNA can be referenced as a moiety in a mobile phase (typically fluid), to be detected by probes (“target probes”) which are bound to the substrate at the various regions.

Biopolymer arrays (e.g., polynucleotide microarrays) can be fabricated by depositing previously obtained biopolymers (such as from synthesis or natural sources) onto a substrate, or by in situ synthesis methods. Methods of depositing obtained biopolymers include, but are not limited to, loading then touching a pin or capillary to a surface, such as described in U.S. Pat. No. 5,807,522 or deposition by firing from a pulse jet such as an inkjet head, such as described in PCT publications WO 95/25116 and WO 98/41531, and elsewhere. The in situ fabrication methods include those described in U.S. Pat. No. 5,449,754 for synthesizing peptide arrays, and in U.S. Pat. No. 6,180,351 and WO 98/41531 and the references cited therein for polynucleotides, and may also use pulse jets for depositing reagents. Further details of fabricating biopolymer arrays by depositing either previously obtained biopolymers or by the in situ method are disclosed in U.S. Pat. Nos. 6,242,266, 6,232,072, 6,180,351, and 6,171,797. In fabricating arrays by depositing previously obtained biopolymers or by in situ methods, typically each region on the substrate surface on which an array will be or has been formed (“array regions”) is completely exposed to one or more reagents. For example, in either method the array regions will often be exposed to one or more reagents to form a suitable layer on the surface that binds to both the substrate and biopolymer or biomonomer. In in situ fabrication the array regions will also typically be exposed to the oxidizing, deblocking, and optional capping reagents. Similarly, particularly in fabrication by depositing previously obtained biopolymers, it can be desirable to expose the array regions to a suitable blocking reagent to block locations on the surface at which there are no features from non-specifically binding to target.

Determining the amount of miRNAs can alternatively, or in addition to microarray analysis, comprise using real-time polymerase chain reaction (PCR), for example such as is disclosed in detail in the present Examples. Real-time PCR (RT-PCR) can provide accurate and rapid data as to presence and amount of miRNAs present in a sample.

In some embodiments, the methods of the invention comprise providing a biological sample from a subject. The biological sample can be a bodily fluid such as described herein, e.g., plasma or serum. An amount of one or more of the miRNAs is then determined and compared to one or more miRNA control levels. The subject can then be diagnosed with having or being at risk of disease if there is a measurable difference in the amount of the one or more miRNAs as compared to the one or more miRNA control levels. The levels of the one or more miRNAs can also be used to provide a prognosis or a theranosis, such as to classify the subject as a likely responder or non-responder to a treatment or to monitor the efficacy of a treatment over time. As such, in some embodiments, methods can include predicting response to a treatment in a subject, or predicting non-response of a treatment in a subject. The control levels can be the levels of the one or more miRNAs in a control sample that does not have or is not at risk of having disease, e.g., the control sample can be from a healthy subject. When monitoring one or more miRNA levels over time, a control can also be the level of the one or more miRNAs at a different time point. For example, a decrease in the level of one or more miRNA in a subject over time may indicate a response to a treatment.

The presently-disclosed subject matter is inclusive of uses of reagents as described herein and reagents known to those of ordinary skill in the art to carry out the methods as disclosed herein and described in the claims. The presently-disclosed subject matter further includes kits that include reagents as described herein and reagents known to those of ordinary skill in the art to carry out the methods as disclosed herein and described in the claims.

The presently-disclosed subject matter further includes systems and kits, which are useful for practicing embodiments of the methods as described herein. In some embodiments a kit is provided, which is useful for determining a presence or an amount of one or more micro miRNAs, which includes a probe for determining the presence or amount of each of one or more mircroRNAs in a sample. In some embodiment, the probe(s) are polynucleotides. In some embodiments a primer pair is used to determine the amount of the one or more miRNAs. In some embodiments, the probe(s) is provided on a substrate. In some embodiments the kit includes a probe for each of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, or 85 miRNAs.

In some embodiments, kits of the presently-disclosed subject matter further include a reference standard sample to obtain a presence or amount of the one or more miRNAs for use as a control to which the sample (e.g., sample from the subject) can be compared. In some embodiments, the systems further include control data of a presence or level of the one or more miRNAs for use as a control to which the sample (e.g., sample from the subject) can be compared. In some embodiments, the systems further include reference data for one or more clinicopathologic features useful for characterizing a disease-of-interest.

In some embodiments, the standard sample or the control data can be selected from: a standard sample or control data for a disease; a standard sample or control data for chronic kidney disease; a standard sample or control data for lupus; a standard sample or control data for cardiometabolic disease; a standard sample or control data for an autoimmune disease; a standard sample or control data for artherosclerosis; a standard sample or control data for a responder; and a standard sample or control data for a nonresponder.

The practice of the presently disclosed subject matter can employ, unless otherwise indicated, conventional techniques of cell biology, cell culture, molecular biology, transgenic biology, microbiology, recombinant DNA, and immunology, which are within the skill of the art. Such techniques are explained fully in the literature. See e.g., Molecular Cloning A Laboratory Manual (1989), 2nd Ed., ed. by Sambrook, Fritsch and Maniatis, eds., Cold Spring Harbor Laboratory Press, Chapters 16 and 17; U.S. Pat. No. 4,683,195; DNA Cloning, Volumes I and II, Glover, ed., 1985; Oligonucleotide Synthesis, M. J. Gait, ed., 1984; Nucleic Acid Hybridization, D. Hames & S. J. Higgins, eds., 1984; Transcription and Translation, B. D. Hames & S. J. Higgins, eds., 1984; Culture Of Animal Cells, R. I. Freshney, Alan R. Liss, Inc., 1987; Immobilized Cells And Enzymes, IRL Press, 1986; Perbal (1984), A Practical Guide To Molecular Cloning; See Methods In Enzymology (Academic Press, Inc., N.Y.); Gene Transfer Vectors For Mammalian Cells, J. H. Miller and M. P. Calos, eds., Cold Spring Harbor Laboratory, 1987; Methods In Enzymology, Vols. 154 and 155, Wu et al., eds., Academic Press Inc., N.Y.; Immunochemical Methods In Cell And Molecular Biology (Mayer and Walker, eds., Academic Press, London, 1987; Handbook Of Experimental Immunology, Volumes I-IV, D. M. Weir and C. C. Blackwell, eds., 1986.

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

In certain instances, microRNAs (miRNAs) disclosed herein are identified with reference to names assigned by the miRBase Registry (available at www.mirbase.org). The sequences and other information regarding the identified miRNAs as set forth in the miRBase Registry are expressly incorporated by reference as are equivalent and related miRNAs present in the miRBase Registry or other public databases. Also expressly incorporated herein by reference are all annotations present in the miRBase Registry associated with the miRNAs disclosed herein. Unless otherwise indicated or apparent, the references to the Sanger miRBase Registry are references to the most recent version of the database as of the filing date of this Application (i.e., mirBase 21, released June 2014).

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments±20%, in some embodiments±10%, in some embodiments±5%, in some embodiments±1%, in some embodiments±0.5%, and in some embodiments±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

The presently-disclosed subject matter is further illustrated by the following specific but non-limiting examples. The following examples may include compilations of data that are representative of data gathered at various times during the course of development and experimentation related to the present invention.

### EXAMPLES

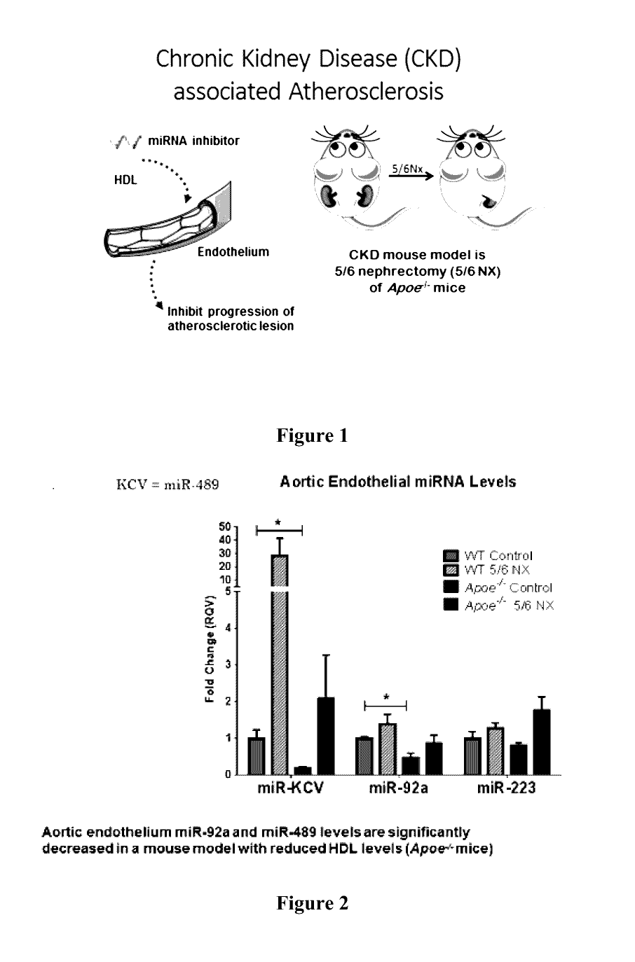

Animal data provided in the following examples provides proof-of-concept and shows 33% reduction in plaque 7 days after IV injection of lipoprotein-associated locked nucleic acids targeting miR-92a and miR-489. Endothelium miR-92a and miR-489 are increased in CKD-associated atherosclerosis model in mice. Administration of miRNA inhibitors to the miR-92a and miR-489 significantly reduces endothelium miR92a and miR489 levels and atherosclerosis progression. HDL mediated delivery of miRNA inhibitor designed to specifically inhibit the miRNA to the endothelium was performed in chronic kidney disease mouse model. (FIG. 1)

### Example 1

Altered HDL-miRNAs in chronic kidney disease subjects compared to control subjects. miRNA was isolated from Wild-type (WT) and apolipoprotein E−/− (Apoe−/−) mouse aortic endothelium by Qiazol perfusion. miR-489 (KCV), miR-92a, and miR-223 levels were quantified in aortic endothelium miRNA by real-time PCR. miR-489 and miR-92a levels were increased in both WT and Apoe−/− aortic endothelium after ⅚ nephrectomy (⅚Nx) compared to controls without ⅚ Nx. Comparing the change in miR-92a and miR-489 in the mouse model with reduced HDL levels (Apoe−/− mice) with and without ⅚ Nx to wild type (WT) mice shows miR-489 and miR-92a levels were increased in both wild type and Apoe−/− aortic endothelium after ⅚ Nx compared to controls without ⅚ Nx. (FIG. 2).

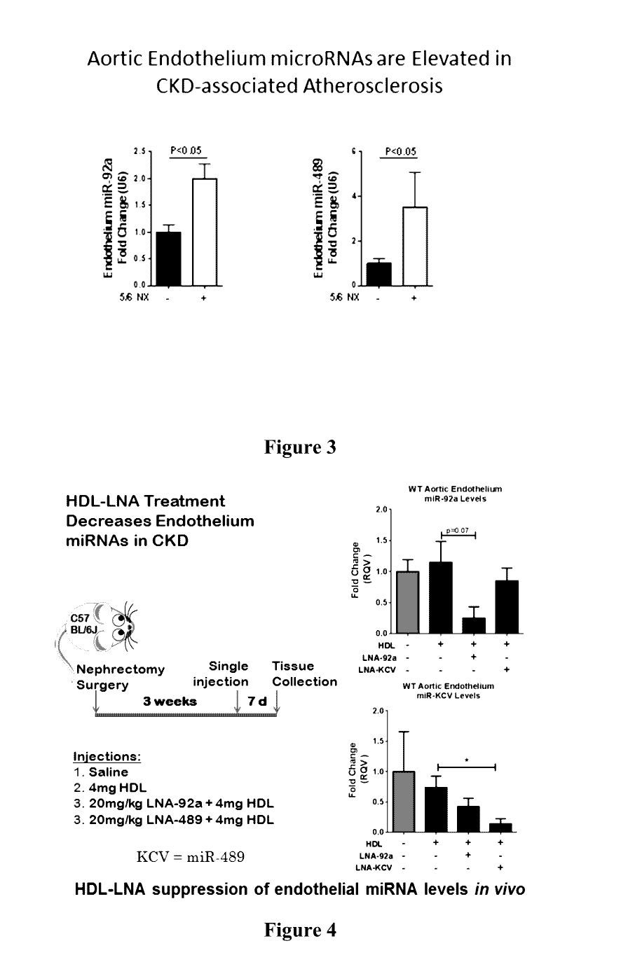

FIG. 3 includes quantification of miR-92a and miR-489 in the aorta endothelium for Apoe^{-/-} mice with sham or ⅚ nx surgeries. Utilizing the same data of FIG. 2, the graph of FIG. 3 demonstrates more specifically the difference in the Apoe−/− mouse background. Results were based on Real-time PCR showing elevation of the miRNAs in chronic kidney disease-associated atherosclerosis, and significance was determined by Mann-Whitney nonparametric test.

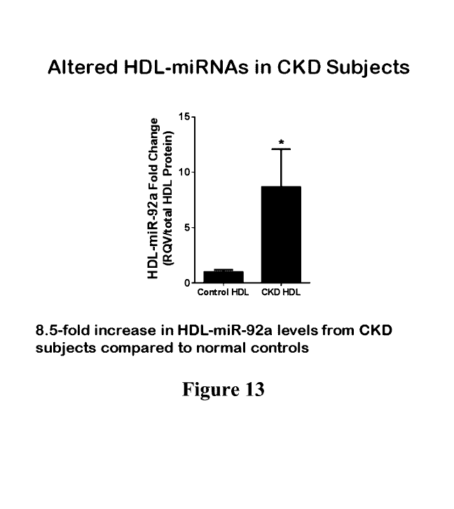

As shown in FIG. 13, HDL-miR-92a levels were found to be 8.5-fold higher in human HDL from CKD subjects compared to control subjects. miR-92a was quantified by real-time PCR. A ⅚ nephrectomy was performed on mice to provide a model of mouse chronic kidney disease. For an example of nephrectomy procedure, see, C M Shing, et al. Effect of Tocopherol on Atherosclerosis, Vascular Function, and Inflammation in Apolipoprotein E (Apoe) Knockout Mice with Subtotal Nephrectomy, Cardiovascular Therapeutics, 2014, incorporated in its entirety.

### Example 2

Reduction in miRNA levels after injections of HDL complexed with locked-nucleic acid inhibitors against miR-92 or miR489 after nephrectomy in WT mice

As shown in FIG. 4, nephrectomy surgery was performed on mice. Three weeks later, a single injection of either saline, 4 mg HDL, 20 mg/kg LNA-92a+4 mg HDL, or 20 mg/kg LNA-489+4 mg HDL of the retro-orbital plexus was performed. 7 days after injection, tissue was collected. RNA was isolated from Wild-type (WT) mouse aortic endothelium by Qiazol perfusion. miR-489 (KCV) and miR-92a levels were quantified in aortic endothelium RNA by real-time PCR. miR-489 and miR-92a levels were decreased in WT mice with ⅚ nephrectomy (⅚ Nx) after injections (retro-orbital plexus) with HDL complexed with locked-nucleic acid inhibitors against miR-92 or miR-489 (4 mg+20 mg/kg locked nucleic acid) compared to saline (control) or HDL (4 mg) injections. *P<0.05.

### Example 3

Inhibition of miR-92a, miR-489, or a Combination of both miR92a and miR-489

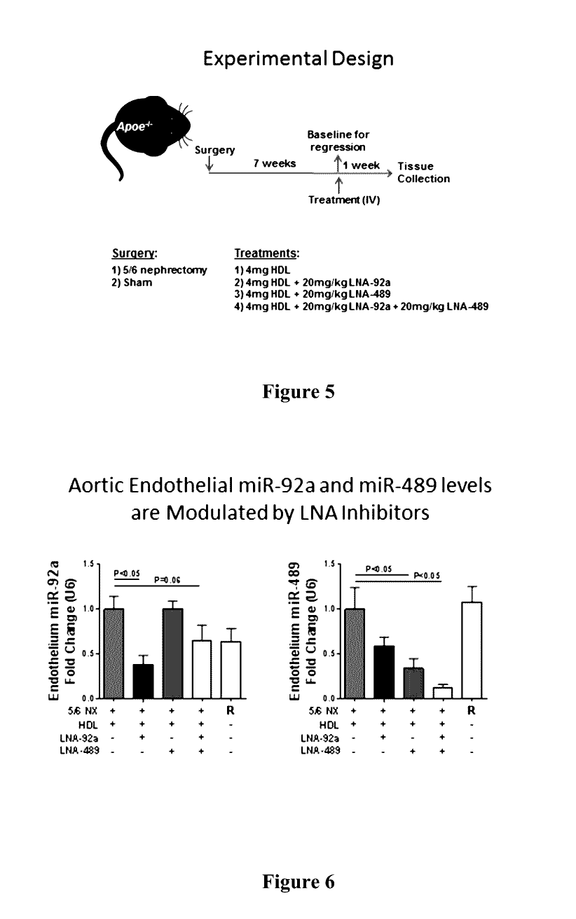

Experimental study diagram outlines the time course of the experiment, FIG. 5. Apoe^{-/-} mice undergo surgery, sham or ⅚ nx, and over a 7 week period the mice develop more severe atherosclerotic lesions due to the decrease in kidney function. At 7 weeks post, a subset of mice are taken as baseline control to measure the atherosclerotic lesion at the start of the treatment. At this 7 week time point, the other mice are treated with a single intravenous injection of 4 mg HDL, 4 mg HDL+20 mg/kg LNA-92a, 4 mg HDL+20 mg/kg LNA-489, or 4 mg HDL+20 mg/kg LNA-92a+20 mg/kg LNA-489. The mice are sacrificed for tissue collection 1 week after this single treatment.

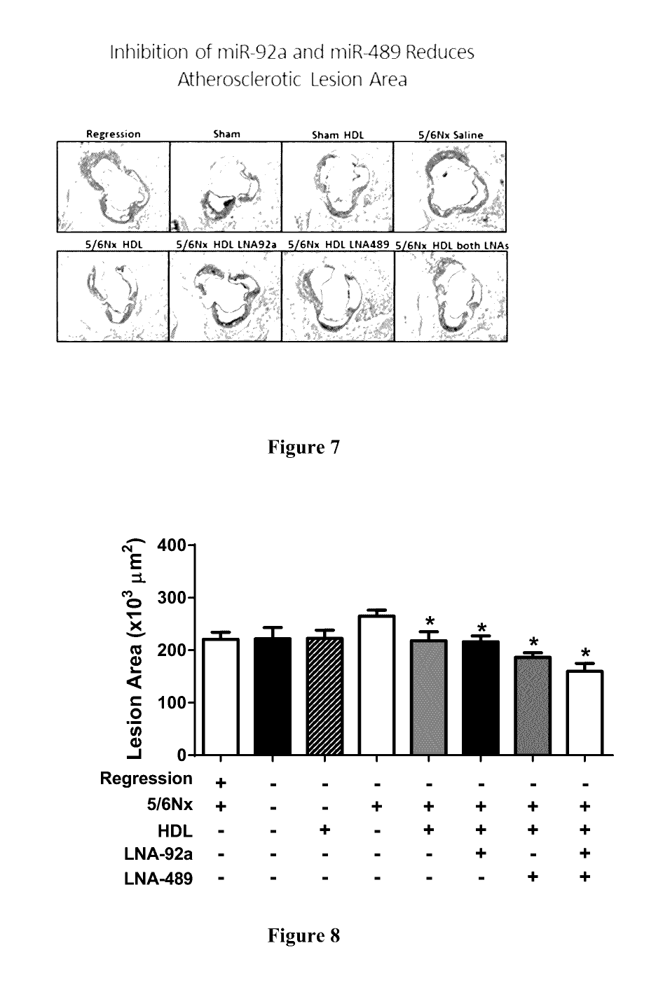

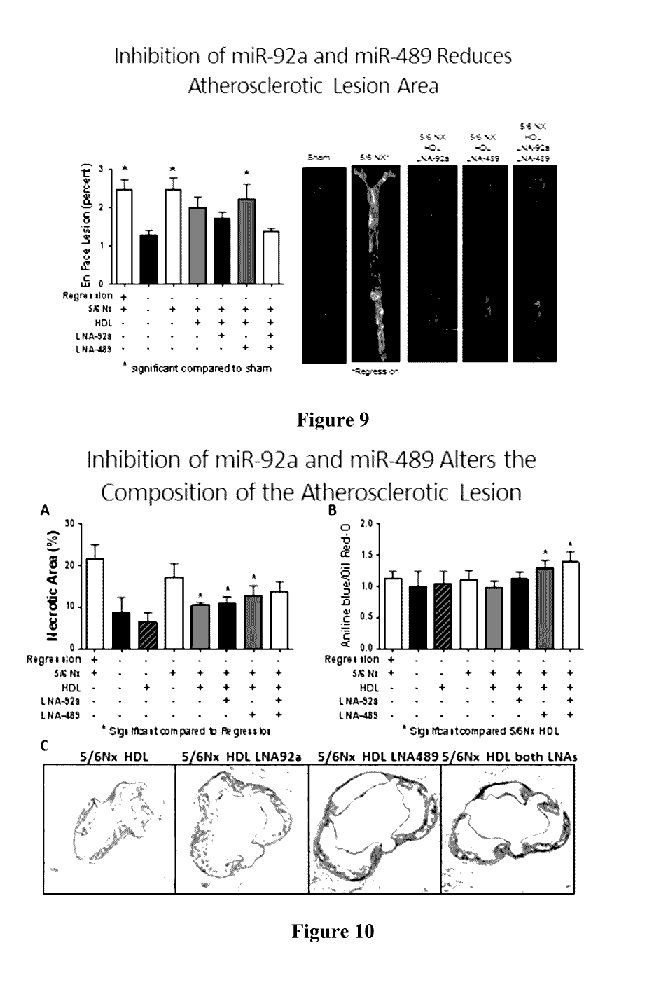

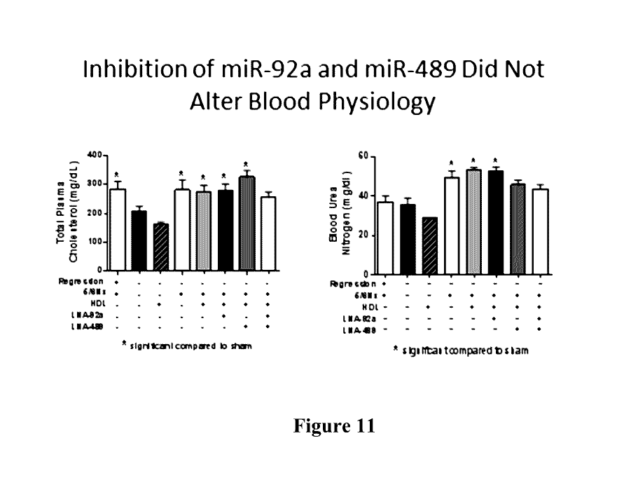

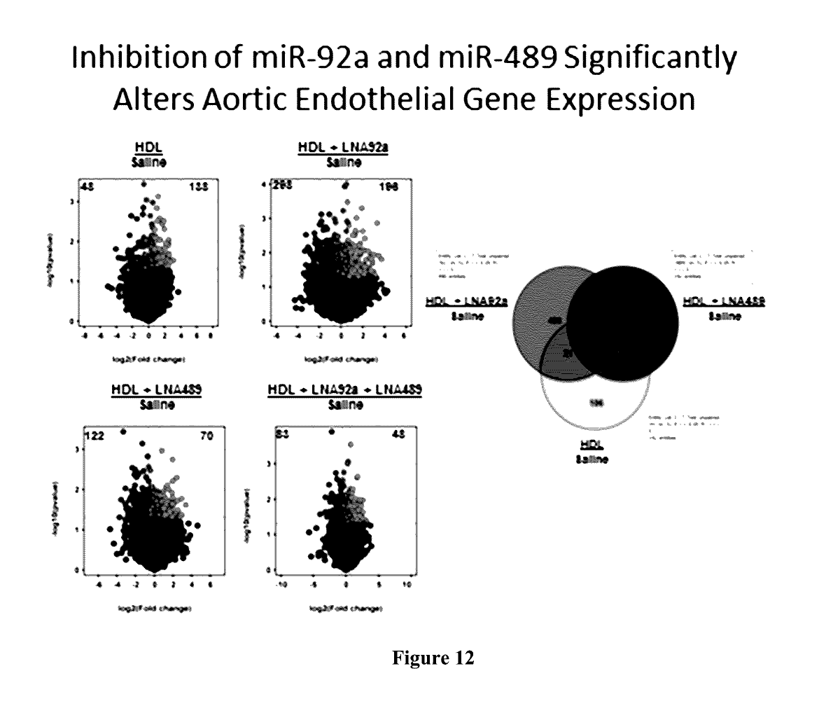

Aortic Endothelial miR-92A and miR-489 levels are modulated by linked nucleic acid (LNA) inhibitors. Real-time PCR quantification of miR-92a and miR-489 in the aorta endothelium of HDL+/−LNA treated Apoe^{-/-} mice demonstrate that the LNAs reduce the miRNAs in the endothelium. N=6-13. Statistical significance determined by one-way ANOVA. (FIG. 6). As can be seen in cross sections of the aortic sinus stained with oil red o (FIG. 7) to visualize the neutral lipids within the atherosclerotic lesions, inhibition of miR-92a and miR-489, alone or in combination, reduces the atherosclerotic lesion area. As shown in FIG. 8, the total atherosclerotic lesion area is reduced when miR-92a and miR-489 are inhibited, with the largest reduction in the administration of both LNA-92a and LNA-489. To further explore this reduction, the aortas from a small cohort were stained with Sudan IV en face to quantify the lesions throughout the length of the aortas. The lesion area as a percent of the whole aorta was quantified with representative images for each experimental group. (FIG. 9). The composition of the atherosclerotic lesion is altered when miR-92a and miR-489 are inhibited (FIG. 10). Statistically significant lesion collagen content increase occurred with the administration of both LNA-489 alone and in combination with LNA-92a, FIG. 10b. The aniline blue stained images are representative images for each experimental group, FIG. 10c. Interestingly, blood physiology was not altered by inhibition of miR-92a and miR489 (FIG. 11). Aortic endothelial gene expression is significantly altered with inhibition of miR-92a and miR489, as shown in the volcano plots of FIG. 12.

The investigators have provided herein a showing that endothelium lipoprotein associated miR-92a and miR-489 are increased in CKD-associated atherosclerosis model. Because CKD subjects have increased cardiovascular events and accelerated atherosclerosis, the CKD subject was an appropriate model to evaluate treatment efficacy of lipoprotein associated miRNA inhibitors. HDL-miRNA communication is altered in chronic kidney disease and contributes to atherosclerosis, and can be manipulated for the treatment of subjects. Administration of miRNA inhibitors significantly reduces endothelium miR-92a and miR-489 levels and atherosclerosis progression, without alteration of blood physiology or HDL cholesterol levels. The experiments show the use of lipoprotein associated miR inhibitors inhibit atherosclerosis progression in vivo and induce lesion regression.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

What is claimed is:

1. A method of treating a condition associated with increased atherogenesis, comprising: administering an effective amount of an agent that inhibits miR-489 or a combination of miR-489 and miR-92a to a subject having the condition or having been identified as being at risk of developing the condition.

2. The method of claim 1, wherein the miRNA is associated with a lipoprotein selected from the group consisting of HDL, LDL, VLDL and subfractions of lipoprotein.

3. The method of claim 1, wherein the condition is selected from atherosclerosis, dyslipidemia, chronic kidney disease, diabetes, an autoimmune disorder, such as lupus, and a cardiometabolic disease associated with increased atherogenesis and cardiovascular disease.

4. The method of claim 1, wherein the subject has or is at risk of developing atherosclerosis

5. The method of claim 1, wherein the agent is a locked-nucleic acid inhibitor.

6. The method of claim 5, wherein the locked nucleic acid molecule consists of a sequence selected from the group consisting of locked nucleic acid anti-miR-489 and locked nucleic acid antimiR-92a.

7. The method of claim 1, wherein the agent is one or more non-naturally occurring nucleic acid molecules that is an anti-miRNA oligonucleotide, which reduces miR-489 expression, miR-92a expression or a combination thereof.

8. The method of any claim 1, wherein the agent is a nucleic acid molecule comprising at least 10, 11, 12, 13, 14, 15, 16, 17, 18, 19, 20, or 21 consecutive nucleotides of the complement of a sequence selected from the group consisting of SEQ ID NO: 1 and SEQ ID NO:2.

9. The method of claim 1, wherein expression of the miRNA(s) is down-regulated.

10. The method of claim 1, wherein expression of the miRNA(s) is down-regulated in vascular endothelial cells.

11. The method of claim 10, wherein expression of the miRNA(s) is down-regulated in vascular tissues.

12. The method of claim 1, and further comprising diagnosing the condition in the subject prior to administering the agent, said diagnosing comprising: (a) obtaining a blood or plasma sample from the subject; and (b) isolating lipoprotein from the blood or plasma.

13. The method of claim 12, and further comprising detecting whether miR-489 and/or miR-92a are present in the isolated lipoproteins.

14. The method of claim 13, and further comprising diagnosing the subject with the condition when miR-489 and/or miR-92a are present in the isolated lipoproteins at increased levels relative to a control.

12. A method of detecting lipoprotein-associated miRNAs in a sample, comprising: contacting the sample with a probe that specifically-binds each of the one or more lipoprotein-associated miRNAs, and determining an amount of each of the probe-bound miRNAs, wherein the lipoprotein is selected from HDL, LDL, and VLDL and subfractions of lipoprotein, and the one or more lipoprotein-associated miRNAs are selected from the group consisting of miR-489 and miR-92a.

13. The method of claim 12, wherein the sample is a lipoprotein sample isolated from a blood or plasma sample from a subject.

14. The method of claim 13, wherein the subject has or is at risk of having atherosclerosis.

15. The method of claim 12, wherein the probe comprises a nucleic acid molecule comprising at least 10, 11, 12, 13, 14, 15, 16, 17, 18, 19, 20, or 21 consecutive nucleotides of selected from the group consisting of SEQ ID NO; 1 and SEQ ID NO: 2 or a complement thereof.

16. The method of claim 12, wherein the probe comprises a nucleic acid molecule consisting of a sequence selected from the group consisting of antisense miR-489 and antisense miR-92a.

17. The method of claim 12, wherein the probe further comprises a label.

18. The method of claim 17, wherein the label is a radioisotope, a fluorescent label, a chemiluminescent label, or an enzymatic label.

19. The method of claim 13, and further comprising administering to the subject an agent that inhibits one or more miRNA selected from miR-489,or a combination of miR-489 and miR-92a, when the subject is predicted as having or being at risk of developing the condition.

20. The method of claim 12, wherein the miRNA is associated with a lipoprotein selected from the group consisting of HDL, LDL, VLDL, and subfractions of lipoprotein.


SEQUENCE LISTING (2 sequences)

SEQ ID NO: 1 - RNA, 22 nt, Homo sapiens
gugacaucacauauacggcagc

SEQ ID NO: 2 - RNA, 22 nt, Homo sapiens
uauugcacuugucccggccugu